(19) United States
(12) Patent Application Publication
(10) Pub. No.: US 2025/0074867 A1
(43) Pub. Date: Mar. 6, 2025

(54) NOVEL CATIONIC LIPID COMPOUNDS

(71) Applicant: Guangzhou Anobri Pharmaceutical Co. Ltd., Guangdong (CN)

(72) Inventors: Cai Gu Huang, Guangdong (CN); Tie Qiang Huang, Guangdong (CN)

(21) Appl. No.: 18/294,314

(22) PCT Filed: Mar. 31, 2022
(86) PCT No.: PCT/CN2022/084244

§ 371 Date: Feb. 1, 2024

(30) Foreign Application Priority Data
Aug. 13, 2021 (CN) 202110930421.4

(51) Int. Cl. C07C 229/24; A61K 9/51; A61K 48/00; C12N 15/113

(52) U.S. Cl. CPC C07C 229/24; A61K 9/5123; A61K 48/0033; C12N 15/113; C12N 2310/14; C12N 2310/141; C12N 2310/531

(57) ABSTRACT

The present invention relates to lipid compounds that can be used alone or in combination with other lipid components such as neutral lipids, charged lipids, steroids and/or analogues thereof, and/or polymer conjugated lipids to form lipid nanoparticles for delivery of therapeutic and/or prophylactic agents. In some examples, lipid nanoparticles are used to deliver nucleic acids, such as messenger RNA and/or antisense RNA. Methods of using these lipid nanoparticles to treat and/or prevent various diseases are also provided.

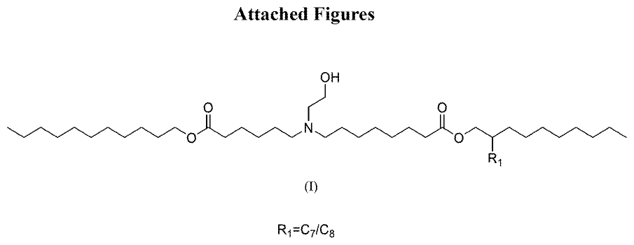

In one embodiment, a compound having the structure of formula (I) below is provided:

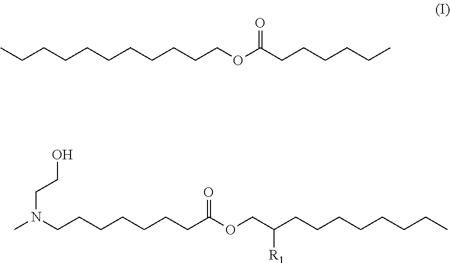

or a salt or isomer thereof or an N-oxide thereof, wherein RI is as defined herein.

Pharmaceutical compositions comprising one or more compounds of the aforementioned structural formula (I) and therapeutic and/or prophylactic agents are also provided. In some embodiments, the pharmaceutical composition further comprises one or more components selected from neutral lipids, charged lipids, steroids, and polymer conjugated lipids. These compositions are useful for forming lipid nanoparticles to deliver therapeutic and/or prophylactic agents.

In other embodiments, the invention provides a method of administering a therapeutic agent and/or prophylactic agent to a subject in need thereof, the method is that pharmaceutical composition comprising lipid nanoparticles and a therapeutic agent and/or prophylactic agent with a compound of formula (I) was prepared, and delivering the composition to the subject.

### TECHNICAL FIELD

The present invention provides methods using that novel cationic lipids for binding to other lipid components (such as neutral lipids, steroids, and polymer conjugated lipids) to form a nucleic acid mRNA lipid nanoparticle composition, which can be used to deliver one or more therapeutic and/or prophylactic agents to mammalian cells or organs and/or to produce polypeptides in mammalian cells or organs. In addition to the novel lipids, the lipid nanoparticle compositions of the present invention may also comprise one or more cationic and/or ionizable amino lipids in a specific proportion, neutral lipids including polyunsaturated lipids, polymer conjugated lipids, steroids, and/or therapeutic and/or prophylactic agents.

### BACKGROUND TECHNOLOGY

The effective targeted delivery of bioactive substances such as small molecule drugs, proteins, and nucleic acids poses a persistent medical challenge. Specifically, it is difficult to deliever nucleic acids to cells due to the relative instability and low cell permeability of nucleic acids. Therefore, there is a need to develop methods and compositions to facilitate delivery of therapeutic and/or prophylactic agents, such as nucleic acids, to cells.

It has been demonstrated that bioactive substances such as small molecule drugs, proteins and nucleic acids can be efficiently transported into cells and/or intracellular compartments using lipid-containing nanoparticle compositions, liposomes and liposome complexes as transport vehicles. These compositions generally comprise one or more “cationic” lipids, including neutral lipids which contain polyunsaturated lipids (e.g. phospholipids), structural lipids (e.g. steroids), and/or polyethylene glycol-containing lipids (polymer conjugated lipids). Cationic lipids include amine-containing lipids, are easily protonated.

However, the use of oligonucleotides in therapeutic settings currently faces two problems. First, free RNA is readily digested by nuclease in the plasma. Second, the ability of free RNA to enter intracellular compartments where relevant translational mechanisms exist is limited. Lipid nanoparticles formed from cationic lipids in combination with other lipid components such as neutral lipids, cholesterol, PEG, PEGylated lipids, and oligonucleotides have been used to prevent RNA degradation in plasma and promote cellular uptake of oligonucleotides.

There is still a need in the pharmaceutical art for improved cationic lipids and lipid nanoparticles for delivery of oligonucleotides. Preferably, these lipid nanoparticles will exhibit optimized drug delivery as well as protecting the nucleic acids from degradation and clearance in serum, which are suitable for systemic or local delivery and provide intracellular delivery of nucleic acids. In addition, these lipid-nucleic acid particles should be well tolerated and provide an adequate therapeutic index such that patient treatment at an effective dose of nucleic acid is not associated with unacceptable toxicity and/or risk to the patient. The present invention provides these advantages and related advantages.

### PUBLICITY

The present invention provides the following novel compounds and methods involving these compounds:

In a first aspect, the present invention relates to compounds of formula (I) as following:

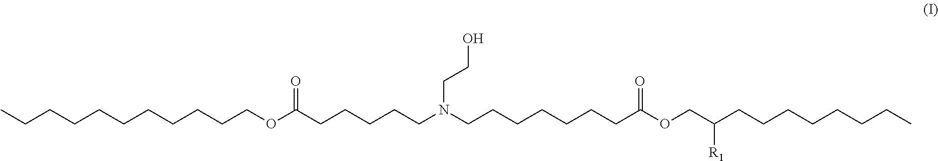

or a salt or isomer thereof or an N-oxide thereof, wherein:
R₁ is a C₇ or C₈ alkyl group.

In various different embodiments, the compound of formula (I) has the representative structures shown below in Table 1.

TABLE 1
Representative Compounds

| Number | Structure |
|---|---|
| A | 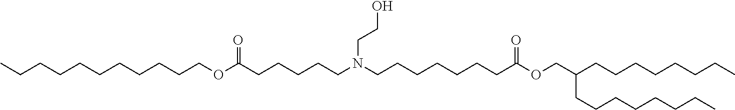 [SMILES: CCCCCCCCCCCOC(=O)CCCCCN(CCO)CCCCCCCC(=O)OCC(CCCCCCCC)CCCCCCCC] |
| B | 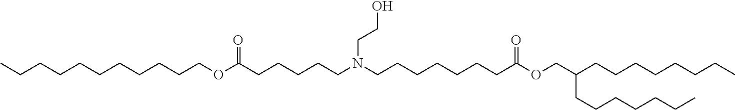 [SMILES: CCCCCCCCCCCOC(=O)CCCCCN(CCO)CCCCCCCC(=O)OCC(CCCCCCC)CCCCCCCC] |

In some embodiments, a composition comprising one or more of the compounds of formula (I) and a therapeutic and/or prophylactic agent is provided.

In some embodiments, the composition comprises one or more of the compounds of formula (I) in combination wtih a therapeutic and/or prophylactic agent. In some embodiments, the composition comprises one compound of formula (I), a therapeutic and/or prophylactic agent, and one or more of the excipients selected from the group consisting of neutral lipids, steroids, and polymer conjugated lipids. Other pharmaceutically acceptable excipients and/or carriers are also included in various embodiments of the composition.

In some embodiments, the neutral lipid is selected from the group consisting of 1,2-dipalmitoyl-sn-glycero-3-phosphocholine (DSPC), 1,2-dipalmitoyl-sn-glycero-3-phosphocholine (DPPC), 1,2-dimethoyl-sn-glycero-phosphocholine (DMPC), 1,2-dioleoyl-sn-glycero-3-phosphocholine (DOPC), 1-palmitoyl-2-oleoyl-sn-glycero-3-phosphocholine (POPC), 1,2-dioleoyl-sn-glycero-3-phosphoethanolamine (DOPE), sphingomyelin (SM), and mixtures thereof. In some embodiments, the neutral lipid is 1,2-distearoyl-sn-glycero-3-phosphocholine (DSPC).

In some embodiments, the steroid is selected from the group consisting of cholesterol, fecal steroid, sitosterol, ergosterol, rapeseed sterol, soybean sterol, rapeseed sterol, tomato base, ursolic acid, alpha-tocopherol, and mixtures thereof. In some embodiments, the steroid is the cholesterol.

In some embodiments, the PEGylated lipid is 1,2-dimyristoyl-sn-glycerol methoxy polyethylene glycol (PEG-DMG).

In some embodiments, the ratio of the components of the composition ranges from about 10 to about 60 mol % of the compound of formula (I), about 0 to about 30 mol % of the neutral lipids, about 10 to about 55 mol % of the steroids, and about 0 to about 10 mol % of the polymer conjugated lipids.

In embodiments of some of the foregoing compositions, the therapeutic and/or prophylactic agents comprise a nucleic acid, wherein the nucleic acid is RNA selected from the group consisting of siRNA, aiRNA, miRNA, dsRNA, shRNA, mRNA, and mixtures thereof. In some embodiments, the RNA is selected from mRNA.

In other embodiments, the invention relates to a method for administering a therapeutic and/or prophylactic agent to a subject in need thereof. In one embodiment, the method includes preparing or providing the composition and administering the composition to the subject.

For administration, the compound of formula (I), typically in the form of lipid nanoparticles in combination with a therapeutic and/or preventive agent, is administered to the subject. The compound of formula (I) is served as active pharmaceutical ingredient or as an ingredient in the form of pharmaceutical dispenses compositions. In one embodiment, the compound of structure (I) is formulated with one or more pharmaceutically acceptable carriers, diluents or excipients to provide the pharmaceutical composition. The compound of structure (I), in the effective form of lipid nanoparticles, delivers therapeutic and/or prophylactic agents. A person of ordinary skill in the art can readily determine suitable concentrations and doses to be administered.

The administration of the pharmaceutical compositions may be carried out using any acceptable mode of administration with any agent for similar utility. The pharmaceutical compositions may be formulated as solid, semi-solid, liquid or gaseous formulation, such as a tablet, capsule, powder, granule, ointment, solution, suspension, suppository, injection, inhaler, gel, microsphere, aerosol, and the like. Typical routes of administration of the pharmaceutical compositions include, but are not limited to, oral, topical, transdermal, inhalation, parenteral, sublingual, oral, rectal, vaginal, and intranasal routes. The term parenteral as used herein includes subcutaneous, intravenous, intramuscular, intradermal, intrasternal, and infusion techniques. A pharmaceutical composition of the invention is formulated so as to allow the active ingredient contained therein to be bioavailable after administration of the pharmaceutical composition to a subject. The pharmaceutical composition to be administered to a subject or patient is in the form of one or more dosage units. In one embodiment, the dosage unit is a tablet that may be a single dosage unit. In one embodiment, the dosage unit is a container containing the compound of formula (I) in aerosol form, wherein the container may contain multiple dosage units. Current methods of preparing these dosage forms are known, or are apparent to those of ordinary skill in the field. In any event, the pharmaceutical composition to be administered will contain a therapeutically effective amount of the compound of formula (I), or a pharmaceutically acceptable salt thereof, in order to treat a relevant disease or condition.

The pharmaceutical composition may be in the form of a solid or liquid. In one embodiment, the carrier is in particle form while the pharmaceutical composition is in tablet or powder form. The carrier may be in the form of a liquid while the composition is an oral syrup or an injectable liquid or aerosol. In one embodiment, the pharmaceutical composition is in the form of an aerosol suitable for inhalation administration.

When intended for oral administration, the pharmaceutical composition is preferably in solid form or liquid form, such as a semi-solid, a semi-liquid, a suspension or a gel form.

As a solid pharmaceutical composition for oral administration, the pharmaceutical composition may be formulated in the form of a powder, granules, compressed tablets, pills, capsules, chewing gums, flakes, and the like. These solid compositions will generally contain one or more inert diluents or edible carriers. In addition, one or more of the following excipient may be included in the pharmaceutical composition: adhesives, such as gelatin, cellulose and the like; excipients, such as lactose and the like; disintegrants, such as alginic acid and the like; lubricants, such as magnesium stearate and the like; glidants, such as silica gel and the like; sweeteners such as sucrose, saccharin and the like; flavoring, such as peppermint and the like; and colorants.

When the pharmaceutical composition is in capsule form, it may contain a liquid carrier, such as polyethylene glycol or oil.

The pharmaceutical composition may be in liquid form, such as syrup, solution, emulsion or suspension. As two examples, liquids may be used for oral administration or for administration by injection. When intended for oral administration, the pharmaceutical compositions may contain, in addition to the compound of formula (I), one or more of sweeteners, preservatives, dyes/colorants, and enhancers. In compositions administered by injection, one or more of a surfactant, a preservative, a wetting agent, a dispersant, a suspension agent, a buffer, a stabilizer, and an isotonic agent may be included in the pharmaceutical composition.

The liquid pharmaceutical composition, whether a solution, suspension, or other similar form, can further include one or more of the following adjuvants: sterile diluents such as water for injection, aqueous brine solution, preferably normal saline, Ringer's solution, isotonic sodium chloride; non-volatile oils such as synthetic monoglycerides or diglycerides, polyethylene glycols, glycerol, propylene glycols, or other solvents that can be used as a dissolving or suspension media; antimicrobial agents, such as nipoquin and vinegar; antioxidants such as ascorbic acid or sodium bisulfite; chelating agents such as ethylene diamine tetraacetic acid; buffering agents such as acetate, citrate and phosphate; and reagents for adjusting tension, such as sodium chloride and glucose; reagents used as cryoprotectants, such as sucrose and trehalose. Parenteral preparations may be packaged in glass or plastic ampoules, disposable syringes, or multi-dose vials. Saline is a preferred adjuvant. The injectable pharmaceutical composition is preferably sterile.

The pharmaceutical composition may consist of dosage units that can be administered as an aerosol. The term aerosol, as used herein, denotes a variety of systems from systems of colloidal nature to systems consisting of pressurized packaging. In aerosol systems, the therapeutic and/or preventive agent may be delivered by liquefied or compressed gas, or by a suitable pump system that disperses the active ingredient. Aerosols of the pharmaceutical compositions may be delivered as monophasic, biphasic, or triphasic systems for delivery of the active ingredient. Systems for the delivery of aerosols includes the necessary containers, activators, valves, sub-containers, and the like, which together can form a kit. Suitable aerosols can be determined by those of ordinary skill in the art without excessive experimentation.

The pharmaceutical compositions can be prepared by methods well known in the pharmaceutical field. Pharmaceutical compositions intended for administration by injection may be prepared by combining the lipid nanoparticles of the invention with sterile distilled water or other carrier to provide a solution. A surfactant may be added to facilitate the formation of a homogeneous solution or suspension. A surfactant is a compound that non-covalently interacts with the compound of formula (I) in order to facilitate dissolution or homogeneous suspension of the compound in an aqueous delivery system.

The pharmaceutical composition or a pharmaceutically acceptable salt is administered in a therapeutically effective amount that will vary according to a variety of factors, including, but not limited to, the activity of the particular therapeutic agent; the metabolic stability and duration of action of the therapeutic agent, the age, weight, general health status, sex and diet of the subject, the mode and time of administration, the excretion rate of the therapeutic agent, other therapeutic agents being co-administered, the seriousness of the disorder being treated, and the like.

The pharmaceutical compositions may also be administered concurrently with, before, or after the administration of one or more other therapeutic agents. These combination therapies include administering a single pharmaceutical dose formulation of the pharmaceutical composition and one or more additional therapeutic agents, and administering the pharmaceutical composition and the other therapeutic agent as individual pharmaceutical dose formulations. For example, pharamaceutical compositions of the invention and the other active agents may be administered to a subject together as a single oral dose composition (e.g., tablets or capsules), or each agent can be administered as different oral dose compositions. When different dosage compositions are used, the pharmaceutical compositions of the invention and the one or more additional therapeutic agents may be administered at substantially the same time, or sequentially at staggered times. It should be understood that combination therapies include all of the above-described methods of administration.

Methods for preparing the compounds of formula (I) and pharmaceutical compositions containing the compounds of formula (I) are described below, and/or are known in the art.

Those of ordinary skill in the art will recognize that in the methods described herein, the functional groups of intermediate compounds used in the preparation of the compounds of formula (I) may need to be protected by suitable protecting groups. These functional groups include, but are not limited to, hydroxyl, amino and carboxylic acid groups. Suitable protecting groups for hydroxyl groups include, but are not limited to, trialkylsilyl or diarylalkylsilyl, tetrahydrofuranyl, benzyl and the like. Suitable protecting groups for amino groups include, but are not limited to tert-butoxycarbonyl, benzyloxycarbonyl, and the like. Suitable protecting groups for carboxylic acids include, but are not limited to, hydrocarbon, aryl, arene esters, and the like. Protecting groups may be added or removed in accordance with standard techniques known to those of ordinary skill in the art.

Those of ordinary skill in the art will also recognize that while protected derivatives of the therapeutic and/or preventive agents used in the methods of the invention may not thereby have pharmaceutical activity, they may be administered to mammals and subsequently metabolized in vivo to form pharmacologically active compounds. These derivatives can therefore be described as “prodrugs”. Prodrugs of the compounds of the invention are therefore included within the scope of the invention.

In addition, all compound of formula (I) in the form of a free base or free acid may be converted to pharmaceutically acceptable salts thereof by suitable inorganic or organic base or acid treatment according to methods known to those technicians in the field. Salts of the compounds of the invention can be converted by standard techniques to their free base or acid formation.

The following embodiments are provided for illustrative purposes and are not limiting.

In the following examples, all solvents and reagents are commercially available and used as is, unless otherwise indicated.

The procedure described below can be used for the synthesis of compounds A and B.

The following abbreviations are used herein:
EDC.HCL: 1-Ethyl-(3-dimethylaminopropyl) carboximide hydrochloride
DCM: dichloromethane
DMAP: 4-Dimethylaminopyridine
DIEA: N, N-diisopropylethylamine

### SPECIFIC MODE OF IMPLEMENTATION

### Example 1

### Representative Route

### Synthesis of Compound A: 2-octyldecyl 8-((2-hydroxyethyl) (6-oxo-6-(undecyloxy)hexyl)amino)octanoate

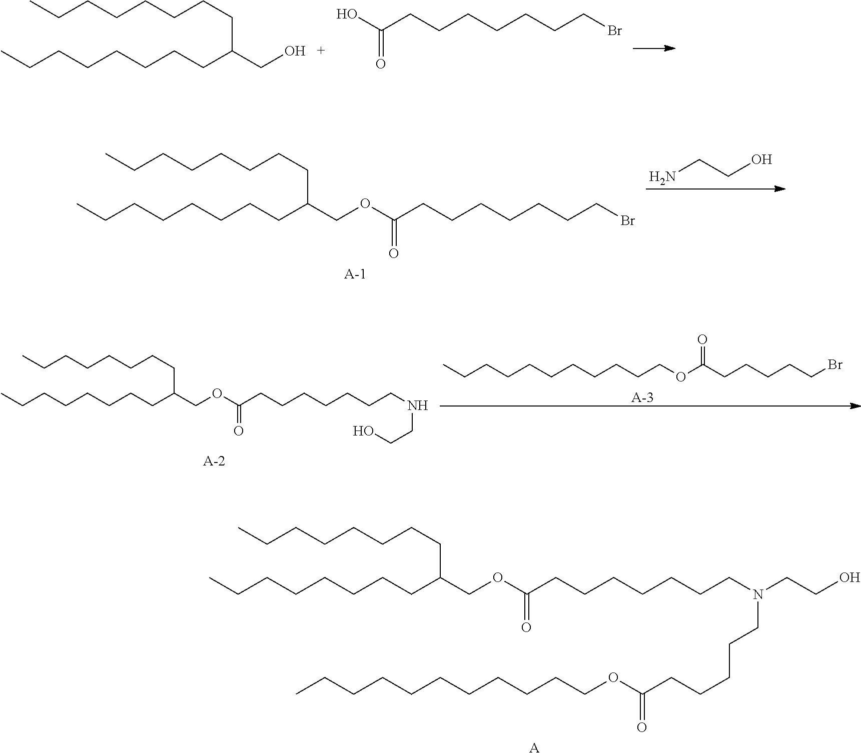

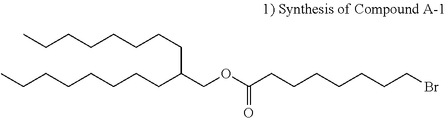 [SMILES: CCCCCCCCC(CCCCCCCC)COC(=O)CCCCCCCBr]

C₂₆H₅₁BrO₂ Chemical Formula:

Molecular Weight: 475.60

To a mixture of 8-bromooctanoic acid (1.0 g, 4.5 mmol) and 2-octyldecanol (1.6 g, 5.8 mmol) in DCM was added EDC.HCl (1.1 g, 5.8 mmol), DIEA (3.3 ml, 18.7 mmol) and DMAP (114 mg, 0.9 mmol) at room temperature. The resulting reaction mixture was stirred for 24 h. After dilution with DCM, the reaction mixture was washed with a saturated aqueous solution of sodium bicarbonate followed by washing with a dilute hydrochloric acid solution, dried over magnesium sulfate, filtered and concentrated, and the resulting residue purified on a silica gel column (0-15% ethyl acetate/n-hexane) to provide compound A-1 (951 mg, 2.0 mmol, 44% yield).

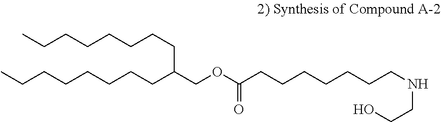 [SMILES: CCCCCCCCC(CCCCCCCC)COC(=O)CCCCCCCNCCO]

C₂₈H₅₇NO₃ Chemical Formula:

Molecular Weight: 455.77

To a solution of Compound A-1 (2.9 g, 6.0 mmol) in 3 mL of ethanol was added 2-aminoethanol (15 ml, 248 mmol). The resulting reaction solution was refluxed at elevated temperature for 2 hours. The reaction mixture was then concentrated under vacuum, dissolved in ethyl acetate, and washed with water. The organic phase was separated, dried over anhydrous sodium sulfate, and concentrated to afford a residue. The residue was purified on a silica gel column (0-100% (mixture of 1% NH₄OH, 20% MeOH in dichloromethane) dichloromethane) to afford compound A-2 (2.4 g, 5.3 mmol, 89% yield).

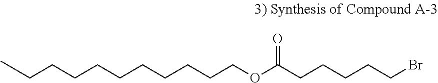 [SMILES: CCCCCCCCCCCOC(=O)CCCCCBr]

C₁₇H₃₃BrO₂ Chemical Formula:

Molecular Weight: 349.35

Compound A-3 can be prepared according to the synthetic method used to prepare compound A-1.

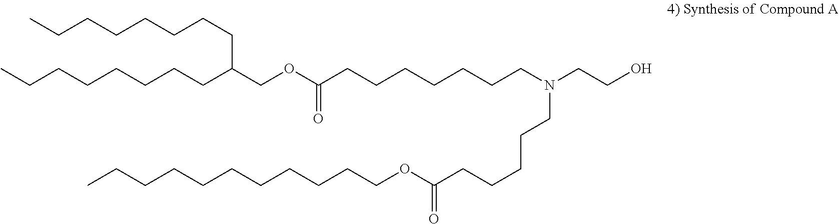 [SMILES: CCCCCCCCCCCOC(=O)CCCCCN(CCO)CCCCCCCC(=O)OCC(CCCCCCCC)CCCCCCCC]

C₄₅H₈₉NO₅ Chemical Formula:

Molecular Weight: 724.21

To an acetonitrile mixture of Compounds A-2 (1.0 g, 2.2 mmol) and A-3 (1.1 g, 3.2 mmol) was added DIEA (414 mg, 3.2 mmol). The resulting reaction mixture was heated to 65° C. and stirred for 24 h. After concentrating the reaction mixture under reduced temperature and pressure, the resulting residue was dissolved in ethyl acetate. The organic phase was washed with a saturated saline aqueous solution, dried over anhydrous sodium sulfate, and concentrated under vacuum to provide a residue. The residue was purified on a silica gel column (0-100% (mixture of 1% NH₄OH, 20% MeOH in dichloromethane) dichloromethane) to afford Compound A (797 mg, 1.1 mmol, 50% yield). C₄₅H₈₉NO₈, Ms m/z: [M+H]⁺724.7; ¹H-NMR (300 MHz, CDCl₃) δ: ppm 4.10 (2H, t), 3.97 (2H, d), 3.67˜3.47 (2H, m), 2.76˜2.36 (5H, m), 2.28 (4H, m), 1.88˜1.41 (15H, m), 1.38˜1.17 (50H, m), 0.87 (9H, m).

### Example 2

### Compound B: Synthesis of 2-heptylnonyl 8-((2-hydroxyethyl) (6-oxo-6-(undecyloxy)hexyl)amino)octanoate

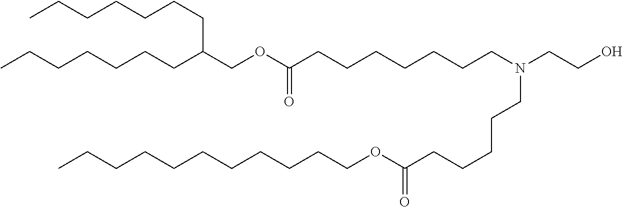 [SMILES: CCCCCCCCCCCOC(=O)CCCCCN(CCO)CCCCCCCC(=O)OCC(CCCCCCC)CCCCCCC] C₄₄H₈₇NO₅ Chemical Formula:

Molecular Weight: 710.18

Compound B can be synthesized according to a representative route described in Example 1.

C₄₄H₈₇NO₅, Ms m/z: [M+H]⁺711.0; ¹H-NMR (300 MHz, CDCl₃) δ: ppm 4.11 (2H, t), 3.98 (2H, d), 3.68˜3.47 (2H, m), 2.76˜2.35 (5H, m), 2.27 (4H, m), 1.88˜1.40 (15H, m), 1.37˜1.17 (48H, m), 0.88 (9H, m).

### Example 3

### In Vivo Evaluation of Luciferase mRNA Using Lipid Nanoparticle Compositions

Cationic lipids, DSPC, cholesterol, and PEG-lipids were dissolved in ethanol in molar ratios of 50:10:37:2 or 48:10:42:2. Lipid nanoparticles (LNPs) were prepared in a total lipid to mRNA weight ratio of about 10:1 to 30:1. mRNA was diluted to 0.15 mg/ml in 10 to 50 ml citrate buffer (pH=4). Using a syringe pump, an ethanolic solution of lipids was mixed with an aqueous mRNA solution in a ratio of approximately 1:5 to 1:3 (volume/volume) at a total flow rate of more than 15 ml/min. Then ethanol was removed and the external buffer replaced by dialysis with PBS. Finally, the lipid nanoparticles were filtered through a 0.2 um pore size sterile filter. The particle size of lipid nanoparticles as determined by quasi-elastic light scattering using Malvern Zetasizer Nano ZS was approximately 65-105 nm in diameter and, in some cases, approximately 75-100 nm in diameter.

The in vivo study was conducted on female mice aged 6-8 weeks and mice aged 8-10 weeks according to guidelines developed by the National Council for Science and Technology. Different doses of mRNA lipid nanoparticles were administered systemically via tail vein injection and the animals euthanized at specific time points (e.g., 5 hours) after dosing. The liver and spleen were collected in pre-weighed tubes, their weights determined, frozen immediately in liquid nitrogen, and stored at −80° C. until use for analysis.

For the liver, approximately 50 mg of liver tissue was cut for analysis and placed in 2 mL FastPrep tubes (MP Biomedicals, Solon OH). ¼″ ceramic beads (MP Biomedicals) and 500 μL of Glo Lysis Buffer-GLB (Promega, Madison WI) were added to each tube and the contents equilibrated to room temperature. The liver tissue was then homogenized for 15 seconds at 2×6.0 m/s using a FastPrep24 instrument (MP Biomedicals). The homogenates were incubated for 5 minutes at room temperature followed by a ¼ dilutions in GLB and evaluated using the SteadyGlo luciferase assay system (Promega). Specifically, 50 μL of diluted tissue homogenate was reacted with 50 μL of SteadyGlo substrate, shaken for 10 seconds, followed by incubation for 5 minutes and quantified using a CentroXS3LB 960 photometer (Berthold Technologies, Germany). The amount of protein was determined using a BCA Protein Assay Kit (Pierce, Rockford IL). Relative luminescence units (RLU) were then normalized to the total ug of the measured protein. To convert RLU into ng luciferase, a standard curve was generated with QuantiLum recombinant luciferase (Promega).

FLuc mRNA (L-6107) from Trilink Biotechnologies will express luciferase protein, and was originally isolated from fireflies (Photinus pyralis). Fluc is commonly used in mammalian cell cultures to measure gene expression and cell viability. It emits biologic light in the presence of the substrate luciferin. This capped and polyadenylated mRNA was completely replaced by 5-methylcytidine and pseudouridine.

### Example 4

### Determination of the pKa of Prepared Lipids

The formulated cationic lipid pKa correlates with the effect of LNPs used to deliver nucleic acids. The preferred pKa range is 5 to 7. The pKa of each cationic lipid was determined in lipid nanoparticles using fluorescence analysis based on 2-(p-toluidine)-6-naphthalenesulfonic acid (TNS). As described in Example 3, lipid nanoparticles containing cationic lipids/DSPC/cholesterol/PEG lipids (50/10/40/1.5 mol %) at a concentration of 0.4 mM total lipid in PBS were prepared using an ordered method. 100 μM stock solution was prepared with TNS in distilled water. The vesicles were diluted to contain 24 μM lipid in 2 mL of a buffer solution containing 10 mM HEPES, 10 mM MES, 10 mM acetic acid, 130 mM NaCl, and having a pH of 2.5 to 11. The equal TNS solution was added to produce a final concentration of 1 uM, and after vortex mixing, fluorescence intensity was measured in a SLM Aminco Series 2 luminescence spectrophotometer at room temperature using excitation and emission wavelengths of 321 nm and 445 nm. Fluorescence data were analyzed using a sigmoidal best fit and pKa was measured as the pH producing half-maximal fluorescence intensity.

### Example 5

The efficacy of the lipid nanoparticle formulations containing various cationic lipids was determined with the rodent models of luciferase mRNA expression in vivo.

The cationic lipids shown in Table 2 were previously tested with nucleic acids. For comparative purposes, these lipids were also used to formulate lipid nanoparticles containing FLuc mRNA (L-6107) using an ordered (in line) mixing method as described in Example 3. Lipid nanoparticles were formulated using the following molar ratios: 50% cationic lipid/10% distearylphosphatidylcholine (DSPC)/40% cholesterol/1.5% PEG lipid (“PEG-DMG”, i.e., (1-(monomethoxy-polyethylene glycol)-2,3-dimyristoylglycerol, average PEG molecular weight 2000). As described in Example 3, relative activity was determined by measuring luciferase expression in the liver 5 hours after administration via tail vein injection. The activity was compared at 0.3 and 1.0 mg mRNA/kg and expressed as ng luciferase/g liver measured 5 hours after administration as described in Example 3.

TABLE 2
Comparison of lipids with mRNA exhibiting activity

| | | Liver Luc @ | Liver Luc @ | |
|---|---|---|---|---|
| | | 0.3 mg/kg | 1.0 mg/kg | Structure |
| Compound | pKa | dose | dose | |
| A | 6.01 | 156 ± 50 | 550 ± 200 | 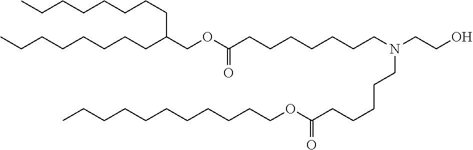 [SMILES: CCCCCCCCCCCOC(=O)CCCCCN(CCO)CCCCCCCC(=O)OCC(CCCCCCCC)CCCCCCCC] |
| B | 5.98 | 134 ± 40 | 522 ± 210 | 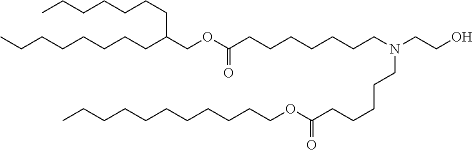 [SMILES: CCCCCCCCCCCOC(=O)CCCCCN(CCO)CCCCCCCC(=O)OCC(CCCCCCC)CCCCCCCC] |

Each of the technical features of the above described embodiments may be combined in any combination. All possible combinations of each of the technical features in the above embodiments are not described in order to make the description concise, however, as long as there is no contradiction in the combination of these technical features, it should be considered within the scope of this specification.

The above described embodiments express only several embodiments of the invention, which are described in more specificity and detail, but should be understood as not limiting the scope of the invention. It should be noted that for those of general technical skill in the art, a number of improvements may also be made without departing from the inventive concept, all of which fall within the scope of the invention.

1. Compounds have the following structure (I): 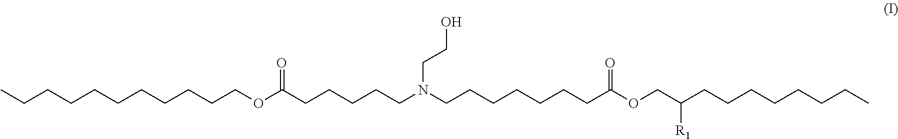
or a salt or isomer thereof or an N-oxide thereof,
wherein R₁ is C₇ or C₈ alkyl.

2. The compound of claim 1 has the following structure (A).

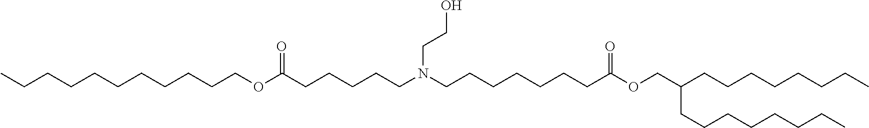 [SMILES: CCCCCCCCCCCOC(=O)CCCCCN(CCO)CCCCCCCC(=O)OCC(CCCCCCCC)CCCCCCCC]

3. The compound of claim 1 has the following structure (B).

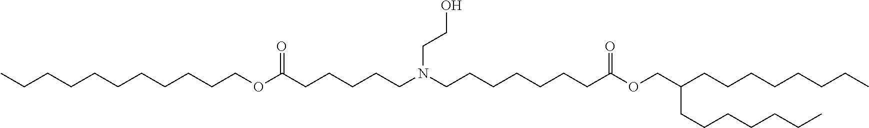 [SMILES: CCCCCCCCCCCOC(=O)CCCCCN(CCO)CCCCCCCC(=O)OCC(CCCCCCC)CCCCCCCC]

4. The composition comprises any one of a compound in claims 1 to 3 and a therapeutic and/or prophylactic agent.

5. The composition of claim 4, further comprising one or more excipients selected from neutral lipids, steroids, and polymer conjugated lipids.

6. The composition of claim 5, wherein the neutral lipid in the component is selected from the group consisting of one or more of 1,2-distearoyl-sn-glycero-3-phosphocholine (DSPC), 1,2-dipalmitoyl-sn-glycero-3-phosphocholine (DPPC), 1,2-dimethoyl-sn-glycero-phosphocholine (DMPC), 1,2-dioleoyl-sn-glycero-3-phosphoch oline (DOPC), 1-palmitoyl-2-oleoyl-sn-glycero-3-phosphocholine (POPC), 1,2-dioleoyl-sn-glycero-3-phosphoethanolamine (DOPE), and sphingomyelin (SM).

7. The composition of claim 6, wherein the neutral lipid is DSPC.

8. The composition of the preceding claims 5 to 7, wherein the steroid in the component is selected from the group consisting of cholesterol, fecal steroid, sitosterol, ergosterol, rapeseed sterol, bean sterol, tomato base, ursolic acid, alpha-tocopherol.

9. The composition of claim 8, wherein the steroid is cholesterol.

10. The composition of the preceding claims 5 to 9, wherein the polymer-conjugated lipid in the component is a pegylated lipid.

11. The composition of claim 10, wherein the PEGylated lipid is 1,2-dimyristoyl-sn-glycerol methoxypolyethylene glycol (PEG-DMG).

12. The composition of any one of the preceding claims, wherein the therapeutic and/or prophylactic agents are vaccines or compounds capable of eliciting an immune response, including nucleic acids.

13. The composition of claim 12, wherein the nucleic acid is RNA selected from the group consisting of siRNA, aiRNA, miRNA, dsRNA, shRNA, mRNA, and mixtures thereof.

14. The composition of claim 13, wherein the RNA is an mRNA.

15. A method is that a therapeutic and/or prophylactic agent was administered to a subject in need thereof, included comprising preparing or providing the composition of any one of the preceding claims and administering the composition to the subject.

16. The subject of any one of the preceding claims is a mammal or a human.
